(19) United States
(12) Patent Application Publication
(10) Pub. No.: US 2010/0017972 A1
(43) Pub. Date: Jan. 28, 2010

(54) BIODEGRADABLE POLYMERIC DERIVATIVES

(76) Inventors: Luciana Sartore, Marano Vicentino (IT); Maurizio Penco, Brescia (IT); Andrea Sassi, Brescia (IT); Manuela Cinzia Candido, Arzignano (IT)

Correspondence Address: NIXON & VANDERHYE, PC, 901 NORTH GLEBE ROAD, 11TH FLOOR, ARLINGTON, VA, 22203, US

(21) Appl. No.: 12/520,231

(22) PCT Filed: Dec. 17, 2007
(86) PCT No.: PCT/IB07/55167

§ 371 Date: Jul. 28, 2009

(30) Foreign Application Priority Data
Dec. 19, 2006 (IT) RM2006A000682

(51) Int. Cl. A61K 38/01; C08J 9/00; D06P 3/32

(52) U.S. Cl. 8/436; 530/407; 521/52

(57) ABSTRACT

The present invention relates to a new class of adducts containing peptidic-based segments of proteic hydrolysates and gelatins, chemically bound to polymeric-based segments of polyethylene glycol (PEG) with variable length and processes for the preparation thereof. The new materials can also contain segments of acrylic or vinylic polymers.

The present invention relates to a new class of adducts containing peptidic-based segments, of proteic hydrolysates and gelatins, chemically bound to polymeric-based segments of polyethylenglycols (PEG) with variable length and processes for the preparation thereof. The new materials can also contain segments of acrylic or vinylic polymers.

### FIELD OF THE INVENTION

The present invention relates to proteic hydrolysate polymer and gelatin-polymer adducts, to processes for the preparation thereof and to the use of said adducts in the industrial field of the plastic materials and in particular for the production of packagings, of culture protection means and of disposable materials of daily use and in the tanning field. More particularly, the invention relates to proteic hydrolysate PEG (polyethylenglycols) adducts and gelatin-PEG adducts, to which acrylic or vinylic polymers can be stapled. In these last materials the acrylic or vinylic polymer is bound to the proteic hydrolysate or to the gelatin by means of a spacer arm constituted by PEG with variable length.

### STATE OF ART

Proteic hydrolysates, which are constituted by mixtures of free amino acids, peptides and oligopeptides, belong to the state of art.

The products obtainable by hydrolysis of materials which can be designated as products, by-products, waste, residues of animal and/or vegetable origin are several; they differentiate for the composition of the free and total amino acids, for the content of free amino acids, for the distribution of the molecular weights and of the average molecular weight. The starting material, the hydrolysability feature of each peptidic bond and the different degradability of the free amino acids, typical of each hydrolysis reaction environment, determines the great variety of potentially available proteic hydrolysates.

Some manufacture processes, used for obtaining proteic hydrolysates produced from the by-products and/or waste and/or residues of the tanning cycle obtained before or after the tanning phase, are described in the Italian patent application 85511/A/82 and in the European patent application with publication number EP1021958A1 in the name of the Applicant.

The same productive processes used for processing by-products and/or waste and/or residues in the working of tanning leathers, can be entirely or partially used for the processing of products, by-products, waste or residues of animal or vegetable origin of the agro-food industry.
The technical progress and the continuous increase in the energetic costs have made convenient and usable innovative techniques such as, for example, the use of enzymes for the hydrolysis and of all techniques useful for improving the organolectic properties (for example, colour, odour) and chemical features (for example saline content, distribution of molecular weights).

The gelatins obtained from by-products and/or waste and/or residues of the tanning cycle obtained before and after the tanning phase, where mainly mild hydrolysis treatments are carried out, belong to the state of art.

In particular, the proteic hydrolysates obtained from by-products and residues of the leather working are appreciated in the agricultural field for the bio-stimulating properties.

The uses of the proteic hydrolysates in the industrial field are several; in particular Manzo and Fedele (1994 and 1996) have studied the possibility of using such products in the tanning field as such or as copolymers with methyl metacrylate or acrylonitrile, in tanning and finishing (Cuo. Pelli. Mater. 1989, 378-391).

### DESCRIPTION OF THE INVENTION

The object of the present invention is a process for the production of adducts of formula (1)

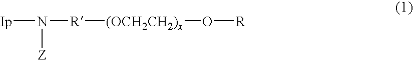

comprising the reaction in water or aqueous solvent of a proteic hydrolysate or gelatin of formula and/or the mixture thereof of formula (3)

IpNH₂ (3)

with a compound of formula (4)

R—(OCH₂CH₂)ₓ—OR (4)

or another bifunctional or multifunctional compound of the family of the epoxides wherein represent
Ip the aminoacidic, peptidic or polypeptidic residue of the proteic hydrolysate or gelatin;
Z —H or -R′—(OCH₂CH₂)ₓ—O—R;
x an integer smaller than 250;

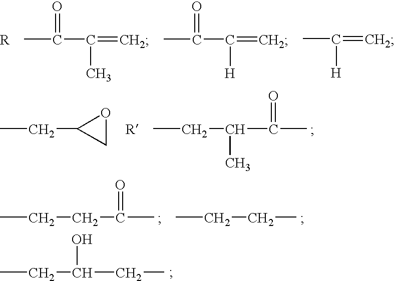

According to the present invention, the proteic hydrolysate and/or gelatin of formula (3) has a ratio between α-aminic nitrogen and organic nitrogen lower than or equal to 0.5 and an average molecular weight comprised between 200 and 100,000. Furthermore, said hydrolysate and/or gelatin is preferably obtained from by-products and/or waste and/or residues coming from the tanning industry obtained before and after the tanning phase or from by-products and/or products of vegetable origin, agro-industrial scraps, by-products and/or products of animal origin.

Advantageously in said formula (1), represent

x an integer smaller than 30, preferably the integers 3, 9 or 13

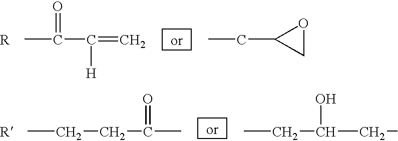

Also the adducts of formula (1) obtainable according to the process shown previously, form an object of the present invention.

### DESCRIPTION OF THE DRAWINGS

Three figures of drawings are enclosed with the present invention which represent:

FIG. 1 a Gel-Permeation chromatography of proteic hydrolysates derivatized with polymers according to the invention;

FIG. 2 the percentage of water absorbed until whole decomposition of films obtained from proteic hydrolysates derivatized with polymers and

FIG. 3 the weight loss until whole decomposition of films obtained from proteic hydrolysates derivatized with polymers.

The adducts of formula (1), which are equally object of the present invention, are typically prepared in water without adding catalyzators and they can be utilized directly.

An additional object of the present invention is a process for the production of cross-linked adducts, wherein the adduct of formula (1)

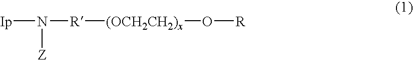

wherein the substituents have the meanings shown previously, is made to react with a condensing agent or a cross-linking agent or a radicalic initiator, with the aim of provoking a reticulation.
Preferably compounds chosen from the class formed by amines, persulfates, peroxides and/or azocompounds are added. The product can reticulate, therefore it becomes insoluble and films with various thickness can be obtained. Such films are biodegradable and the decomposition speed is function of the structure thereof as underlined in the following examples. Also the reticulated adducts obtainable from the process just described and the biodegradable materials obtained from these adducts form an object of the invention.

The products of formula (1) can also be made to copolymerize with acrylic or vinylic monomers.

Therefore, an additional object of the present invention is a process for the production of adducts of formula (2)

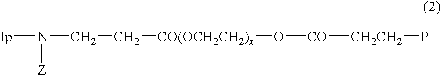

comprising a reaction between a compound of formula (1)

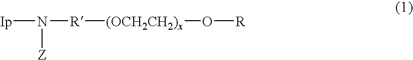

and monomers of acrylic and/or vinylic type of general formula (5)

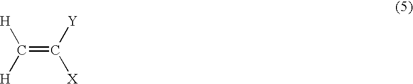

wherein
Ip represents the aminoacidic, peptidic or polypeptidic residue of the proteic hydrolysate or gelatin;
Z corresponds to —H or -R′—(OCH₂CH₂)—O—R or —CH₂—CH₂—CO(OCH₂CH₂)ₓ—O—CO—CH₂CH₂—P;

x is an integer smaller than 250;
R corresponds to

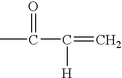

R′ corresponds to

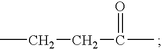

Y corresponds to H, CH₃, Cl, Br or F;
X corresponds to: Cl, Br, F, H,

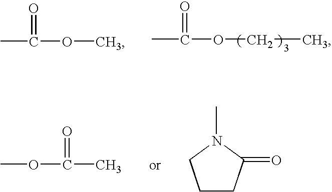

P represents a polymer of acrylic or vinylic type of general formula (6)
   wherein

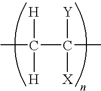

Y corresponds to H, CH₃, Cl, Br or F;
X corresponds to: Cl, Br, F, H,

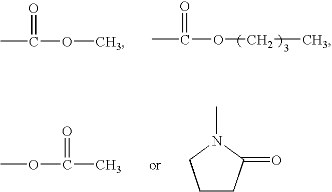

n is an integer smaller than 250.

Advantageously, said proteic hydrolysate and/or gelatin of formula (3) has a ratio between a-aminic nitrogen and organic nitrogen lower than or equal to 0.5 and an average molecular weight comprised between 200 and 100,000.

The hydrolysate and/or gelatin are preferably obtained from by-products and/or waste and/or residues coming from the tanning industry obtained before and after the tanning phase or from by-products and/or products of vegetal origin, agro-industrial scraps, by-products and/or products of animal origin. Preferably x represents an integer smaller than 30, more preferably 3 or 10.

Preferably, the polymer of formula (6) is chosen from the class formed by polyvinyl pyrrolidone, polyvinyliden chloride, polyvinyl acetate; and acrylic polymers selected from polymethyl acrylate, polybutyl acrylate, polymethyl metacrylate.

The reaction which leads to the production of compounds of formula (2) is carried out in water or aqueous solvents and it is based upon the co-polymerization of acrylic monomers in presence of proteic hydrolysate and/or gelatin modified with polyethylenglycol diacrylate (of formula 1) and it involves the bond between the aminic groups of the proteic hydrolysate and/or gelatin and the acrylic groups of the polyethylenglycol and it allows obtaining new adducts which form too an object of the present invention.

Vinylic polymers selected among polyvinyl pyrrolidone, polyvinylidene chloride, polyvinyl acetate are preferred; and acrylic polymers selected among polymethyl acrylate, polybutyl acrylate, polymethyl metacrylate.

The adducts of formula [2] equally form an object of the present invention.

Various proteic hydrolysates and gelatins can be used for the preparation of products and/or of the innovative materials object of this patent. The proteic hydrolysates and the gelatins obtained from by-products, waste and residues of the working of tanning leather and the proteic hydrolysates and the gelatins deriving from slaughtering by-products, such as for example the pork rind and the “Cornunghia” already successfully utilized in the agricultural and industrial field, are suitable particularly to be used as proteic basis for the adducts object of the present invention for the convenient quality/price ratio also associated to a great availability on the market. Also hydrolysates of vegetal origin obtained from soya and maize gluten and hydrolysates of animal origin, such as milk, have features which make them advantageously usable for the implementation of the materials object of the present invention. The difference in the aminoacidic composition and in the molecular weights of the proteic hydrolysates and/or gelatins deriving from the mentioned by-products and/or products of vegetal and animal origin, makes it possible the preparation of adducts with various features which could be better suited to the market requirements and to particular operative requests, such as for example affinity with a certain substrate, more suitable mechanical features, compatibility with other materials etc Experimental tests which have been carried out have shown that also the amino acids precipitated from proteic hydrolysates can be used as such or in mixture with the hydrolysates for the implementation of the polymeric derivatives object of the present invention. In particular, the proteic hydrolysates deriving from by-products and/or waste of the working of the tanning cycle are used as such in the re-tanning formulations and in the dyeing and fattening formulations. In the re-tanning phase, they improve the organolectic properties such as softness, fullness, touch and roundness and important physical properties such as the resistance to tear. In the dyeing and fattening phase they improve the dyeing yield, the brightness and homogeneity. The polymers object of the present invention are able to provide improved applicative properties thanks to the increased compatibility, for example, with mixtures of colorants, to the increased reactivity with respect to the proteic hydrolysates used as such and to the possible use in other leather working phases, such as for example the finishing.

An additional object of the present invention is the use of adducts of formula (1), of cross-linked adducts of formula (1), of adducts of formula (2) and of materials obtainable from these adducts in formulations for the re-tanning and the leathers' dyeing and the fattening and for the leathers' finishing.

The polymers object of this patent characterizing for the filming and workability properties can be used advantageously in the industrial field of the plastics for the preparation of products usable in the agricultural, industrial field and in the field of the consumer goods.

In agriculture innovative techniques are used ever more frequently providing the use of materials such as cloths and disks for the mulching, seed cells, pots, tissues and protective nets. For the wrapping and packaging smalls bags, films, nets and strings generally made with the conventional plastic materials are used, which, however, from the environmental point of view have the only advantage of optimizing and/or limiting the use of herbicides, fertilizers, irrigation and manpower. For example, cloths for the mulching have been widely used for years as they allow keeping the optimum humidity in the ground by avoiding dehydration and by guaranteeing the protection from intense cold and the defence from the infestation of weeds by avoiding hoeing. Biodegradable and compostable plastic materials are available on the market, obtained from natural sources and therefore renewable such as, for example, those obtained from cellulose derivatives and in particular from maize starch (Mater-bi of Novamont, Italy) and those obtained from lactic acid by fermentation in particular of maize (NatureWorks PLA of Cargill Dow Polymers LLC, USA). The use of biodegradable and compostable plastic materials involves considerable effects for the environmental safeguard as there is the reduction in the quantity of disposed waste, the recycling of resources, the exploitation of renewable resources with the pollution reduction. For example, in case of use of biodegradable cloths for the mulching, the removal and disposal phase is eliminated and at the same time the cultivation technique is improved as there is a greater compatibility with the soil and the vegetal organisms. The use of biodegradable materials of natural origin confers additional economical advantages such as the cost savings linked to the ground compactation, the drilling, the removal, the disposal and additional agronomic advantages such as the good adhesion to the ground and the improvement in the quality of the ground which remains softer and aired. The use cycle ends up with the milling and/or silting and it starts again with the supply of biodegradable natural residues to the ground useful for the growth of new cultivations.

The technological evolution associated to the experimentation of these materials has made possible the success of materials with higher and higher and more and more competitive performances from the economical point of view, thanks to the implementation thereof at cheaper costs and, from the application point of view, thanks to the implementation thereof with sealing features compatible with the requested time, resistance to the atmospherical agents, mechanical resistance for the mechanization, workability, mouldability, colourability in line with the productive needs and the usual utilized applicative technologies. The materials obtained from the derivatization of proteic hydrolysates and gelatins with polymers allow preparing products alternative to the conventional plastic and to biodegradable and compostable plastic materials existing on the market. They can be used in the industrial, agricultural and consumer-good field and they further have the advantage of having fertilizing and biostimulating qualities which are typical of the proteic hydrolysates. The proper choice of the proteic hydrolysate and/or gelatin and the possible addition of other substances with fertilizing action or other actions, will make the use of these materials in agriculture still more advantageous. For example, in case of cloths for the mulching obtained by using proteic hydrolysates, natural residues remain in the ground, able to provide a fertilizing and biostimulating action. The proteic hydrolysates have a recognized biostimulating activity since they contribute to improve the quality, the productions and the development of the vegetal species. They facilitate the growth and the development of the root apparatus, the biomass, the inflorescences and the fruits in qualitative and quantitative terms; they confer a higher resistance to climatic and physiological stresses, an improved absorption and the use of nutrients. Generally, they improve the performances of all physiological activities involving high productive performances accompanied by consequently decreased supplies of pesticides and supporting inorganic fertilizers.

The materials object of this patent can be used for the production of other materials useful for the agriculture such as for example disks, nets, tissues for the revegetation, pots, seed cells for the agriculture and the forestry plantation, containers for the transplant, strings.

The materials object of this patent can be obtained in solution and/or aqueous or hydroalcoholic suspension. Such formulations allow the direct dispersion onto the ground with the possible “in situ” preparation of films characterized by a resistance and biodegradability which can be modulated depending upon the operability requested by the specific cultures to be protected. The materials can be preferably, but not exclusively, in aqueous or hydroalcoholic solution and they can include pigments and/or reinforcing fibers and/or other materials or substances which make them more suitable to the specific applications. The materials in liquid form can be applied, for example, with the spraying techniques already utilized nowadays for the treatments with pesticides and which can be used with the applicative techniques which are and will be the result of the technological evolution. The mulching carried out with products in liquid form, by allowing the “direct application” onto the ground, has a lower operative cost with respect to the mulching with cloths as it requires less manpower. An additional economic advantage is represented by the fact that the soluble or suspendable products do not need other working phases, such as filming, moulding or extrusion processes. Furthermore, they are more versatile as they are apt to be deposited with variable thicknesses upon use by also allowing the easy additivation of substances such as pigments, reinforcing fibres, fertilizers, pesticides, etc.

The product object of this patent can be used in agriculture also for implementing biodegradable capsules, for example for seeds, or for implementing products and in particular for the microincapsulation of active substances to be used by leaf or root way or directly onto the fruits with the aim of opposing the washout of active substances and for a correct release in time.

An additional object of the present invention is the use of adducts of formula (1), the cross-linked adducts of formula (1), the adducts of formula (2) and the materials which can be obtained from these adducts for the production of materials for the agriculture, such as cloths and disks for the mulching, seed cells, pots, tissues, protective nets and for the production of soluble and/or suspendable materials, to be applied and generated “in situ”.

The materials object of this patent are suitable to be used in the packaging field for the possibility of implementing, for example, sacs, shoppers, bags, nets, strings, cloths with innovative features of biodegradability. The materials object of this patent are suitable to be used for implementing consumer products such as objects, tissues and disposable products.

An additional object of the present invention is the use of adducts of formula (1), the cross-linked adducts of formula (1), the adducts of formula (2) and the materials which can be obtained from these adducts for the production of materials substituting the plastic materials in the field of consumer goods.

### EXAMPLES

In the following examples the term “Ip” designates the proteic hydrolysate or gelatin, whereas the polymers will be referred to with the abbreviations usual for the art. Only some proteic hydrolysates with particular chemical-physical features will be shown.

### A. Modification of Proteic Hydrolysate with Polyethylenglycol

### Example 1

### Sicit1

### Reagents

| | |
|---|---|
| Quantity of proteic basis in g | 400.17 |
| (α-aminic nitrogen (% p/p) = 1.07; dry | |
| substance (% p/p) = 66.4) | |
| Quantity of PEG diacrylate (Mw: 700) in g | 214.65 |
| Quantity of soda (1M) in g | 7.2 |
| Quantity of water in g | 180.32 |

### Methodology

Add the polyethylenglycol diacrylate to the proteic hydrolysate (Ip) and stir for about 10 minutes with mechanical stirrer. Dilute with ⅔ of water by keeping the reaction mixture under stirring. Add NaOH until pH 8 and then the remaining quantity of water.

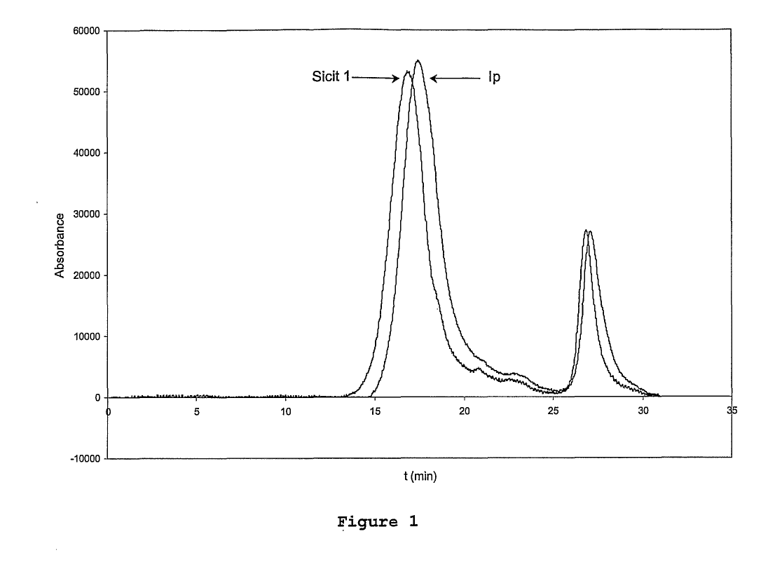

The reaction mixture is to be kept under stirring for about 24 h by controlling the pH, which has to be kept between 7 and 8, in the first reaction hours. The product was characterized by means of IR, NMR, gel permeation chromatography techniques (FIG. 1) and furthermore according to the method for determining the aminic groups by means of reaction of Snyder and Sabocinski with 2,4,6-trinitrobenzensulfonic acid (Snyder S. L. and Sabocinski P. Z., Anal. Biochem. 64, 248-288, 1975), the modification level of the proteic hydrolysate was evaluated (Table 1).

### Example 2

### Sicit2

### Reagents:

| | |
|---|---|
| Quantity of proteic basis in g | 400.43 |
| (α-aminic nitrogen (% p/p) = 1.07; dry | |
| substance (% p/p) = 66.4) | |
| Quantity of PEG diacrylate (Mw: 700) in g | 107.04 |
| Quantity of soda (1M) in g | 5.6 |
| Quantity of water in g | 291.32 |

### Methodology

As described in example 1 it is the addition reaction between PEG diacrylate (Mw 700) and Ip. The samples were prepared with the same methodology but with a lower PEG ratio and consequently a lower modification percentage (Table 1).

### Example 3

### Reagents

| | |
|---|---|
| Quantity of proteic basis in g | 100.1 |
| (α-aminic nitrogen (% p/p) = 1.07; dry | |
| substance (% p/p) = 66.4) | |
| Quantity of PEG diacrylate (Mw: 700) in g | 53.5 |
| Quantity of soda (1M) in g | 1.2 |
| Quantity of water in g | 45.6 |
| Quantity of ammonium persulphate in g | 0.5 |

### Methodology

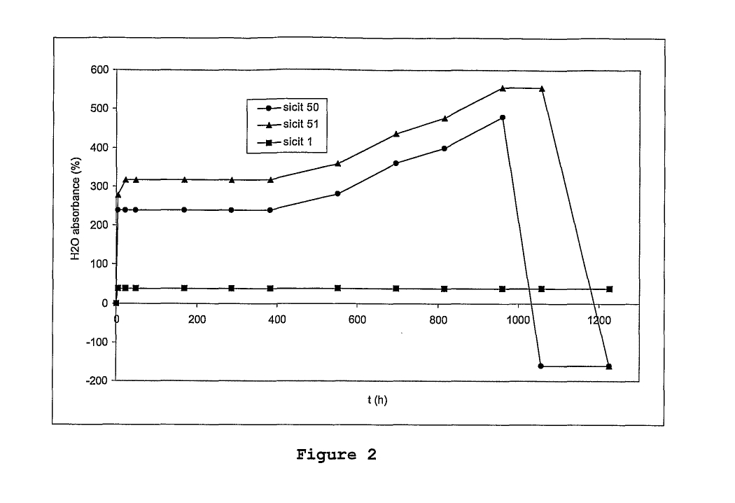

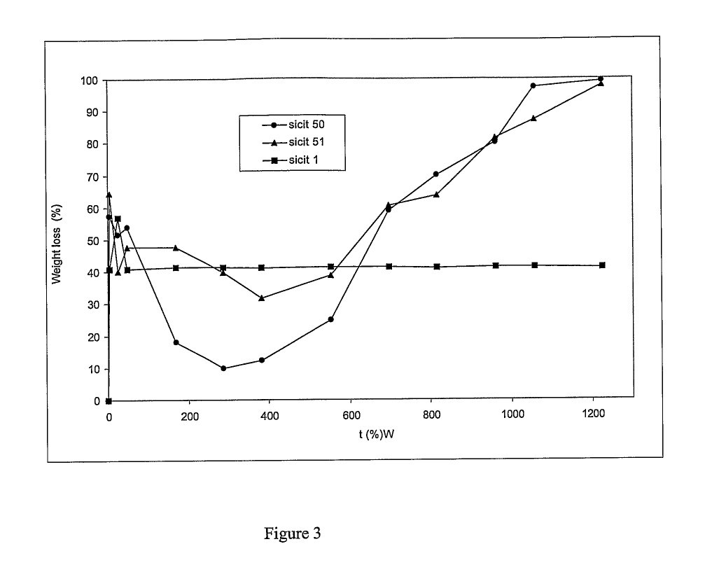

This sample can be obtained by polymerization starting from the sample described in example 1 (and therefore also the one in the example 2) and it shows a very high molecular weight. The methodology described in the example 1 is followed in order to obtain the proteic hydrolysate modified with PEG and then a radicalic initiator such as the ammonium persulphate (or benzoil peroxide) is added to the reaction mixture and the mixture is heated in oil bath at 60° C. for 4 h. As an alternative, the product can be poured, after adding the radicalic initiator, over moulds with suitable sizes and heated in stove at 60° C. The polymerization starts which leads to the production of cross-linked and insoluble products and, in case it has taken place in moulds, films with variable sizes are obtained which reinflate in water and degrade with variable speed (Table 1, FIG. 3, FIG. 2).

### Example 4

### Sicit4

### Reagents:

| | |
|---|---|
| Quantity of proteic basis in g | 404.6 |
| (α-aminic nitrogen (% p/p) = 1.07; dry | |
| substance (% p/p) = 66.4) | |
| Quantity of PEG diglycidyl ether (Mw: 526) in g | 162.35 |
| Quantity of soda (1M) in g | 2.1 |
| Quantity of water in g | 240.1 |

### Methodology

The sample is prepared with the same methodology of the material described in the example 1, but using polyethylenglycol diglycidyl ether instead of PEG diacrylate (Table 1).

### Example 5

### Sicit6

### Reagents:

| | |
|---|---|
| Quantity of proteic basis in g | 406.24 |
| (α-aminic nitrogen (% p/p) = 1.07; dry | |
| substance (% p/p) = 66.4) | |
| Quantity of PEG diglycidyl ether (Mw: 526) in g | 81.63 |
| Quantity of soda (1M) in g | 1.58 |
| Quantity of water in g | 320.53 |
| Quantity of ethylendiamine in g | 2.5 |

### Methodology

Also the proteic derivatives of the polyethylenglycol diglycidyl ether can be cross-linked by condensation or by means of using “hardening” reagents such as, for example, the ethylendiamine. The material is prepared by following the same methodology described in the example 4; after 24 h of reaction under stirring and at room temperature, the trietylamine is added. The materials starts reticulating widely. Also in this case the reticulation can be made in suitable moulds by obtaining films with various thicknesses. (Table 1).

### Example 6

### Sicit 7

### Reagents:

| | |
|---|---|
| Quantity of proteic basis in g | 200.05 |
| (α-aminic nitrogen (% p/p) = 3.26; dry | |
| substance (% p/p) = 71.1) | |
| Quantity of PEG diglycidyl ether (Mw: 526) in g | 40.00 |
| Quantity of soda (1M) in g | 1.6 |
| Quantity of water in g | 158.4 |
| Quantity of ethylendiamine in g | 1.2 |

### Methodology

The proteic derivatives of polyethylen glycol can be obtained starting from proteic hydrolysates with different content of aminic groups and therefore with different molecular weight; the same methodology of example 5 is followed by using a suitable proteic hydrolysate (Table 1).

### Example 7

### Sicit 8

### Reagents:

| | |
|---|---|
| Quantity of proteic basis in g | 100.02 |
| (α-aminic nitrogen (% p/p) = 0.23; dry | |
| substance (% p/p) = 42.12) | |
| Quantity of PEG diglycidyl ether (Mw: 526) in g | 12.79 |
| Quantity of soda (1M) in g | 0.5 |
| Quantity of water in g | 12.88 |
| Quantity of ethylendiamine in g | 0.4 |

### Methodology

The proteic derivatives of polyethylen glycol can be obtained starting from proteic hydrolysates with different content of aminic groups and therefore with different molecular weight; the same methodology of example 5 is followed by using a suitable proteic hydrolysate. (Table 1, FIG. 2 and FIG. 3).

Two samples with the same composition of the example 7, but with different thickness of 1.3 mm and 1.8 mm, respectively called Sicit 50 and Sicit 51, were obtained.

### B. Modification of Proteic Hydrolysate with Acrylic or Vinylic Polymers

### Example 8

### Sicit 16

### Reagents:

| | |
|---|---|
| Quantity of proteic basis in g | 400.22 |
| (α-aminic nitrogen (% p/p) = 1.07; dry | |
| substance (% p/p) = 66.4) | |
| Quantity of PEG diacrylate (Mw: 258) in g | 28.0 |
| Quantity of soda (1M) in g | 11.0 |
| Quantity of water in g | 210.9 |
| Quantity of vinylpyrrolidone in g | 160.01 |
| Quantity of ammonium persulphate in g | 1.6 |

### Methodology

The product was obtained by means of a first functionalization of the proteic hydrolysate with insertion of a spacing arm of PEG diacrylate and a subsequent stapling of PVP.

The proteic base, diluted with about 70 g of H₂O and mixed with mechanical stirrer, is added with polyethylenglycol diacrylate. Soda is added until pH 8. After 24 h under stirring at room temperature, vinylpyrrolidone, the remaining water and the ammonium persulphate are added to the reaction mixture.

The mixture is heated at 60° C. in oil bath for 4 hours by keeping the product under stirring (Table 1).

### Example 9

### Sicit 19

### Reagents:

| | |
|---|---|
| Quantity of proteic basis in g | 228.7 |
| (α-aminic nitrogen (% p/p) = 3.26; Dry | |
| substance (% p/p) = 71.1) | |
| Quantity of PEG diacrylate (Mw: 258) in g | 16.0 |
| Quantity of soda (1M) in g | 6.35 |
| Quantity of water in g | 114.3 |
| Quantity of vinylpyrrolidone in g | 91.4 |
| Quantity of ammonium persulphate in g | 0.92 |

### Methodology

The proteic derivatives of polyvinyl pyrrolidone can be obtained starting from proteic hydrolysates with different content of aminic groups and therefore with different molecular weight; the same methodology followed in the example 8 using a suitable proteic hydrolysate is followed (Table 1).

### Example 10

### Sicit 20

### Reagents:

| | |
|---|---|
| Quantity of proteic basis in g | 100 |
| (α-aminic nitrogen (% p/p) = 0.23; dry | |
| substance (% p/p) = 42.12) | |
| Quantity of PEG diacrylate (Mw: 258) in g | 4.74 |
| Quantity of soda (1M) in g | 2.5 |
| Quantity of water in g | 18.66 |
| Quantity of vinylpyrrolidone in g | 25.36 |
| Quantity of ammonium persulphate in g | 0.25 |

### Methodology

The proteic derivatives of polyvinyl pyrrolidone can be obtained starting from proteic hydrolysates with different content of aminic groups and therefore with different molecular weight; the same methodology followed in the example 8 is followed by using a suitable proteic hydrolysate (Table 1).

### Example 11

### Sicit 21

### Reagents:

| | |
|---|---|
| Quantity of proteic basis in g | 400.22 |
| (α-aminic nitrogen (% p/p) = 1.07; dry | |
| substance (% p/p) = 66.4) | |
| Quantity of PEG diacrylate (Mw: 258) in g | 40.0 |
| Quantity of soda (1M) in g | 20.0 |
| Quantity of water in g | 260.9 |
| Quantity of methylacrylate in g | 80.01 |
| Quantity of benzoyl peroxide in g | 1.6 |

### Methodology

The product was obtained by means of a first functionalization of the proteic hydrolysate with insertion of a spacing arm of PEG diacrylate and a subsequent stapling of methyl acrylate.

The proteic base, diluted with about 70 g of H₂O and mixed with mechanical stirrer, is added with polyethylenglycol diacrylate. Soda is added until pH 8. After 24 h under stirring at room temperature, methyl acrylate, the remaining water and, lastly, the initiator, the benzoyl peroxide are added to the reaction mixture.

The mixture is heated at 70° C. in oil bath for 4 hours by keeping the product under stirring (Table 1).

### Example 12

### Sicit 23

### Reagents:

| | |
|---|---|
| Quantity of proteic basis in g | 400.6 |
| (α-aminic acid (% p/p) = 1.07; dry | |
| substance (% p/p) = 66.4) | |
| Quantity of PEG diacrylate (Mw: 258) in g | 40.4 |
| Quantity of soda (1M) in g | 20.1 |
| Quantity of water in g | 260.0 |
| Quantity of butyl acrylate in g | 80.1 |
| Quantity of benzoyl peroxide in g | 1.6 |

### Methodology

The product was obtained by means of a first functionalization of the proteic hydrolysate with insertion of a spacing arm of PEG diacrylate and a subsequent stapling of butyl acrylate. The same procedure described in the example 11 is followed, by replacing an equal quantity of butyl acrylate to the methyl acrylate. The product is kept in oil bath at 85° C. for 4 hours under stirring (Table 1).

### Example 13

### Sicit 25

### Reagents:

| | |
|---|---|
| Quantity of proteic basis in g | 400.22 |
| (α-aminic nitrogen (% p/p) = 1.07; dry | |
| substance (% p/p) = 66.4) | |
| Quantity of PEG diacrylate (Mw: 258) in g | 40.0 |
| Quantity of soda (1M) in g | 20.0 |
| Quantity of water in g | 260.7 |
| Quantity of MethylMetAcrylate in g | 80.0 |
| Quantity of Benzoyl Peroxide in g | 1.6 |

### Methodology

The product was obtained by means of a first functionalization of the proteic hydrolysate with insertion of a spacing arm of PEG diacrylate and a subsequent stapling of methyl metacrylate. The same procedure described in the example 11 is followed by replacing an equal quantity of methyl metacrylate to the methyl acrylate. The product is kept in oil bath at 80° C. for 4 hours under stirring (Table 1).

### Example 14

### Sicit 27

### Reagents:

| | |
|---|---|
| Quantity of proteic basis in g | 400.26 |
| (α-aminic nitrogen (% p/p) = 1.07; Dry | |
| substance (% p/p) = 66.4) | |
| Quantity of PEG diacrylate (Mw: 258) in g | 40.3 |
| Quantity of soda (1M) in g | 20.0 |
| Quantity of water in g | 260.5 |
| Quantity of VinylAcetate in g | 80.0 |
| Quantity of BenzoylPeroxide in g | 1.6 |

### Methodology

The product was obtained by means of a first functionalization of the proteic hydrolysate with insertion of a spacing arm of PEG diacrylate and a subsequent stapling of vinyl acetate. The same procedure described in the example 11 is followed by replacing an equal quantity of vinyl acetate to the methyl acrylate. The product is kept in oil bath at 70° C. for 4 hours under stirring (Table 1).

### Example 15

### Reagents:

| | |
|---|---|
| Quantity of proteic basis in g | 400.26 |
| (α-aminic nitrogen (% p/p) = 1.07; Dry | |
| substance (% p/p) = 66.4) | |
| Quantity of PEG diacrylate (Mw: 258) in g | 40.2 |
| Quantity of soda (1M) in g | 20.0 |
| Quantity of water in g | 260.6 |
| Quantity of vinylidene chloride in g | 80.0 |
| Quantity of BenzoylPeroxide in g | 1.6 |

The product was obtained by means of a first functionalization of the proteic hydrolysate with insertion of a spacing arm of PEG diacrylate and a subsequent stapling of methyl metacrylate. The same procedure described in the example 11 is followed by replacing an equal quantity of vinylidene chloride to the methyl acrylate. The reaction mixture is kept in reactor at T=70° C., p=50 atm for 4 hours under stirring (Table 1).

### C. Applications

### Example 16

Samples obtained in form of film with a thickness of 1.5 mm were cut out so as to obtain a probe with sizes:

L=3 cm, h=1.5 cm. Such probes, weighed, were plunged into 10 mL of distilled water and thermostated at 24° C. At pre-established times the test pieces and the aqueous phase were measured and weighed. Table 2 and FIG. 2 show the quantity of absorbed water, weight loss and the whole decomposition time for the test pieces obtained according to example 3 and example 7.

TABLE 1
Composition of the materials and evaluation of the modification
of the aminic groups of the proteic hydrolysate

| Example | % p/p | | | | |
|---|---|---|---|---|---|
| Nr. | Composition | | | | —NH₂ |
| (prod. | | | Other | | modif. |
| code) | PEG da | PEG dg | polymer | Ip | (%) |
| 1 | M_{w}: 700 | — | — | α-NH₂: 1.07% | 46 |
| Sicit 1 | 26.7% | | | 33.1% | |
| 2 | M_{w}: 700 | — | — | α-NH₂: 1.07% | 37 |
| (Sicit 2) | 13.3% | | | 33.1% | |
| 3 | M_{w}: 700 | — | — | α-NH₂: 1.07% | 46 |
| Sicit 1 + p | 26.7% | | | 33.1% | |
| 4 | — | M_{w}: 526 | — | α-NH₂: 1.07% | 40 |
| Sicit 4 | | 20.1% | | 33.2% | |
| 5 | — | M_{w}: 526 | | α-NH₂: 1.07% | 20 |
| Sicit 6 | | 10.1% | | 33.3% | |
| 6 | — | M_{w}: 526 | | α-NH₂: 3.26% | 17 |
| Sicit 7 | | 9.99% | | 35.5% | |
| 7 | | M_{w}: 526 | | α-NH₂: 0.23% | 25 |
| Sicit 8 | | 10.1% | | 33.3% | |
| 8 | M_{w}: 258 | — | PVP: 20% | α-NH₂: 1.07% | 13 |
| Sicit 16 | 3.5% | | | 33.1% | |
| 9 | M_{w}: 258 | — | PVP: | α-NH₂: 3.26% | 5 |
| Sicit 19 | 3.5% | | 19.97% | 35.53% | |
| 10 | M_{w}: 258 | — | PVP: | α-NH₂: 0.23% | 30.5 |
| Sicit 20 | 3.12% | | 16.74 | 27.8 | |
| 11 | M_{w}: 258 | — | PMA | α-NH₂: 1.0% | 20.0 |
| Sicit 21 | 4.98% | | 9.97% | 33.1% | |
| 12 | M_{w}: 258 | — | PBA | α-NH₂: 1.07% | 20.1 |
| Sicit 23 | 5% | | 9.98% | 33.13% | |
| 13 | M_{w}: 258 | — | PMMA | α-NH₂: 1.07% | 19.8 |
| Sicit 25 | 4.98% | | 9.97% | 33.1% | |
| 14 | M_{w}: 258 | — | PVAc | α-NH₂: 1.07% | 20.0 |
| Sicit 27 | 5% | | 10% | 33.1% | |
| 15 | M_{w}: 258 | — | PVDC | α-NH₂: 1.07% | 20.0 |
| | 5% | | 9.97% | 33.08% | |

Legend:
PEG da = polyethylenglycol diacrylate (characterized by the molecular weight shown in the table);
PEG dg = polyethylenglycol diglycidyle (characterized by the molecular weight shown in the table);
Ip = Proteic hydrolysate (characterized by the percentege of aminic groups shown in the table);
PVP = polyvinyl pyrrolidone,
PMA = polymethylacrilate;
PBA = polybutylacrylate;
PMMA = polymethylmetacrylate;
PVAc = polyvinyl acetate;
PVDC = polyvinyliden chloride.

1. A process for the production of adducts of formula (1) 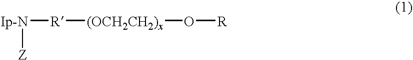
comprising reaction in water or aqueous solvent of a protein hydrolysate or gelatin of formula and/or of mixtures thereof of formula (3)
IpNH₂ (3)
with a compound of formula (4)
R—(OCH₂CH₂)ₓ—OR (4)
or other bi-functional or multi-functional compound of the epoxide family wherein
Ip the aminoacidic, peptidic or polypeptidic residue of the protein hydrolysate or gelatin;
Z —H or -R′—(OCH₂CH₂)ₓ—O—R;
x an integer smaller than 250;

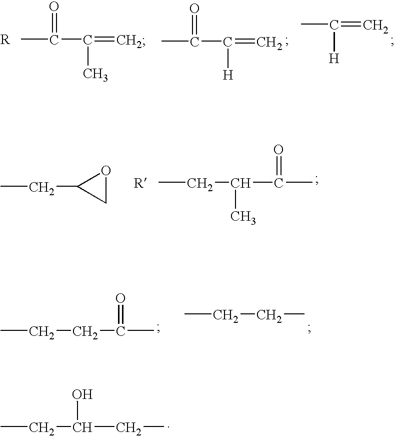

2. The process according to claim 1, wherein said protein hydrolysate and/or gelatin in said formula (1) has a ratio between α-aminic nitrogen and organic nitrogen smaller than or equal to 0.5 and an average molecular weight comprised between 200 and 100,000.

3. The process according to claim 1, wherein said protein hydrolysate and/or gelatin are obtained from by-products and/or waste and/or residues coming from the tanning industry obtained before and after the tanning phase or from by-products and/or products of vegetal origin, agro-industrial scraps, by-products and/or products of animal origin.

4. The process according at claim 1, wherein in said formula (1),
x an integer smaller than 30;

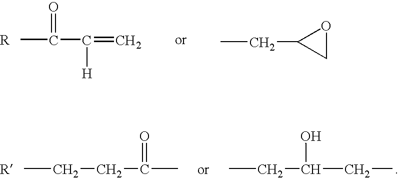

5. The process according to claim 1, wherein in said formula (1) x is 3, 9 or 13.

6. Adduct of formula (1) obtainable according to the process as claimed in claim 1.

7. A process for the production of cross-linked adducts, comprising reacting the adduct of formula (1) 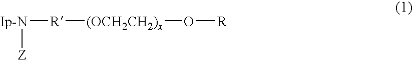
wherein the substituents have the meanings shown in claim 1 a condensing agent or with a cross-linking agent or a radical initiator to perform reticulation.

8. The process according to claim 7, wherein said condensing agent or cross-linking agent or radical initiator is selected from the group consisting of amines, persulfates, peroxides and azo-compounds.

9. Reticulated adduct obtainable from the process as claimed in claim 7.

10. Biodegradable material obtainable from the adduct as claimed in claim 9.

11. A process for the production of adducts of formula (2) 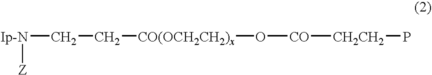
comprising a reaction between a compound of formula (1) 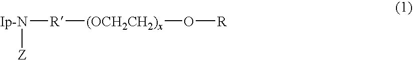
with monomers of acrylic and/or vinyl type of general formula (5) 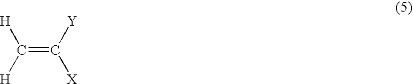
wherein
Ip represents the aminoacidic, peptidic or polypeptidic residue of a protein proteic hydrolysate or gelatin;
Z corresponds to —H or -R′—(OCH₂CH₂)ₓ—O—R or —CH₂—CH₂—CO(OCH₂CH₂)ₓ—O—CO—CH₂CH₂—P;
x is an integer smaller than 250;
R corresponds to 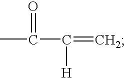
R′ corresponds to 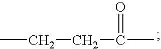
Y corresponds to H, CH₃, Cl, Br or F;
X corresponds to Cl, Br, F, H, or 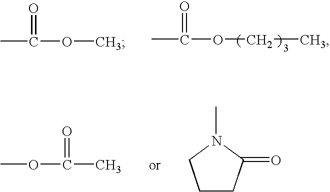
P represents a polymer of acrylic or vinylic type of general formula (6) 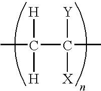
wherein
Y corresponds to H, CH₃, Cl, Br or F;
X corresponds to Cl, Br, F, H, 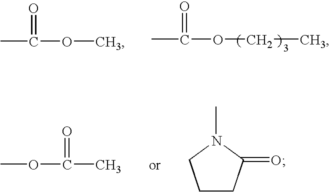
n is an integer smaller than 250.

12. The process according to claim 11, wherein said protein hydrolysate and/or gelatin in said formula (2) has a ratio between α-aminic nitrogen and organic nitrogen lower than or equal to 0.5 and an average molecular weight between 200 and 100,000.

13. The process according to claim 11, wherein said protein hydrolysate and/or gelatin are obtained from by-products and/or waste and/or residues coming from the tanning industry obtained before and after the tanning phase or from by-products and/or products of vegetal origin, agro-industrial scraps, by-products and/or products of animal origin.

14. The process according to claim 11, wherein x represents an integer smaller than 30.

15. The process according to claim 11, wherein in said formula (2) x is 3 or 10.

16. The process according to claim 11, wherein the polymer of said formula (2) is selected from the group consisting of polyvinyl pyrrolidone, polyvinylidene chloride, polyvinyl acetate, polymethyl acrylate, polybutyl acrylate, and polymethyl methacrylate.

17. Adduct obtainable by the process as claimed in claim 11.

18. Use of the adduct as claimed in claim 6 for the production of material for the packaging.

19. Use of the adduct as claimed in claim 6 for the production of the material for the agriculture, such as cloths and disks for the mulching, seed cells, pots and protective cloth nets.

20. Use of the adduct as claimed in claim 6 for the production of material for the agriculture, in soluble and suspendable form, applicable directly onto the ground for the formation of films and cloths for the spray mulching or the mulching carried out with other applicative techniques.

21. Use of the adduct as claimed in claim 6 for the production of the material to be used as substances for coating seeds or for microencapsulating active substances to be used by foliar or radical way or directly onto fruits, for the release modulation.

22. Use of the adduct as claimed in claim 6 for the production of the material substituting plastic materials in the field of the consumer goods.

23. Use of the adduct as claimed in claim 6 in formulation for the leather's retanning and the leathers' dyeing and fattening and for the leathers' finishing.

24. Reticulated adduct obtainable from the process as claimed in claim 8.

25. Biodegradable material obtainable from the adduct as claimed in claim 24.

26. Use of the adduct as claimed in claim 9 for the production of material for the packaging.

27. Use of the adduct as claimed in claim 9 for the production of the material for the agriculture, such as cloths and disks for the mulching, seed cells, pots and protective cloth nets.

28. Use of the adduct as claimed in claim 9 for the production of material for the agriculture, in soluble and suspendable form, applicable directly onto the ground for the formation of films and cloths for the spray mulching or the mulching carried out with other applicative techniques.

29. Use of the adduct as claimed in claim 9 for the production of the material to be used as substances for coating seeds or for microencapsulating active substances to be used by foliar or radical way or directly onto fruits, for the release modulation.

30. Use of the adduct as claimed in claim 9 for the production of the material substituting plastic materials in the field of the consumer goods.

31. Use of the adduct as claimed in claim 9 in formulation for the leather's retanning and the leathers' dyeing and fattening and for the leathers' finishing.

32. Use of the adduct as claimed in claim 17 for the production of material for the packaging.

33. Use of the adduct as claimed in claim 17 for the production of the material for the agriculture, such as cloths and disks for the mulching, seed cells, pots and protective cloth nets.

34. Use of the adduct as claimed in claim 17 for the production of material for the agriculture, in soluble and suspendable form, applicable directly onto the ground for the formation of films and cloths for the spray mulching or the mulching carried out with other applicative techniques.

35. Use of the adduct as claimed in claim 17 for the production of the material to be used as substances for coating seeds or for microencapsulating active substances to be used by foliar or radical way or directly onto fruits, for the release modulation.

36. Use of the adduct as claimed in claim 17 for the production of the material substituting plastic materials in the field of the consumer goods.

37. Use of the adduct as claimed in claim 17 in formulation for the leather's retanning and the leathers' dyeing and fattening and for the leathers' finishing.

38. Use of the adduct as claimed in claim 17 for the production of material for the packaging.

39. Use of the adduct as claimed in claim 17 for the production of the material for the agriculture, such as cloths and disks for the mulching, seed cells, pots and protective cloth nets.

40. Use of the adduct as claimed in claim 17 for the production of material for the agriculture, in soluble and suspendable form, applicable directly onto the ground for the formation of films and cloths for the spray mulching or the mulching carried out with other applicative techniques.

41. Use of the adduct as claimed in claim 17 for the production of the material to be used as substances for coating seeds or for microencapsulating active substances to be used by foliar or radical way or directly onto fruits, for the release modulation.

42. Use of the adduct as claimed in claim 17 for the production of the material substituting plastic materials in the field of the consumer goods.

43. Use of the adduct as claimed in claim 17 in formulation for the leather's retanning and the leathers' dyeing and fattening and for the leathers' finishing.

44. Use of the adduct as claimed in claim 24 for the production of the material for the agriculture, such as cloths and disks for the mulching, seed cells, pots and protective cloth nets.

45. Use of the adduct as claimed in claim 24 for the production of material for the agriculture, in soluble and suspendable form, applicable directly onto the ground for the formation of films and cloths for the spray mulching or the mulching carried out with other applicative techniques.

46. Use of the adduct as claimed in claim 24 for the production of the material to be used as substances for coating seeds or for microencapsulating active substances to be used by foliar or radical way or directly onto fruits, for the release modulation.

47. Use of the adduct as claimed in claim 24 for the production of the material substituting plastic materials in the field of the consumer goods.

48. Use of the adduct as claimed in claim 24 in formulation for the leather's retanning and the leathers' dyeing and fattening and for the leathers' finishing.
